(19) United States
(12) Patent Application Publication
(10) Pub. No.: US 2023/0012963 A1
(43) Pub. Date: Jan. 19, 2023

(54) MEMS AIRBORNE ULTRASONIC TRANSDUCER SYSTEM FOR DETECTING BRAIN HAEMORRHAGE

(71) Applicant: ORTA DOGU TEKNIK UNIVERSITESI, Ankara (TR)

(72) Inventors: Baris BAYRAM, Ankara (TR); Asaf Behzat SAHIN, Ankara (TR)

(73) Assignee: ORTA DOGU TEKNIK UNIVERSITESI, Ankara (TR)

(21) Appl. No.: 17/783,693

(22) PCT Filed: Dec. 9, 2019
(86) PCT No.: PCT/TR2019/051050

§ 371 Date: Jun. 9, 2022

(51) Int. Cl. A61B 5/02; A61B 5/00; B06B 1/02

(52) U.S. Cl. CPC A61B 5/02042; A61B 5/0093; A61B 5/4064; B06B 1/0215; B06B 1/0292; B06B 2201/51; B06B 2201/76

(57) ABSTRACT

An MEMS airborne ultrasonic transducer system operating on a thermoacoustic principle to determine brain haemorrhage, includes: an RF transmitter and ultrasound receiver systems to transmit RF energy and receive ultrasound wave, respectively, an RF transmitter system having an RF signal generator, an RF amplifier and a horn antenna, and an ultrasound receiver system having a lock-in amplifier, a DC supply and two ultrasonic transducer arrays wirebonded to low noise amplifier (LNA) chips. The MEMS airborne ultrasonic transducer system determines brain haemorrhage based on detecting RF-induced, blood-originating, thermoacoustic ultrasound wave at the pulse modulation frequency.

### CROSS REFERENCE TO THE RELATED APPLICATIONS

This application is the national phase entry of International Application No. PCT/TR2019/051050, filed on Dec. 9, 2019, the entire contents of which are incorporated herein by reference.

### TECHNICAL FIELD

The invention relates to MEMS airborne ultrasonic transducer system to detect thermoacoustic generation of ultrasound wave caused by the RF-induced volumetric expansion of blood in the brain.

### BACKGROUND

Developing diseases related to brain haemorrhage today negatively affect the peace and prosperity of the society. Detection within the first 1.5 hours after the onset of cerebral haemorrhage has enabled treatment and prevention of permanent brain damage effectively reducing the healing time. However, detection using magnetic resonance imaging (MRI) or computed tomography (CT) can hardly be completed in this time frame due to high cost and complexity of the equipment limiting their readiness and availability.

Stroke is the third most significant disease in the world after heart disease and lower respiratory tract infection, and stroke is the first neurological disease among the world in terms of all the negative burden caused by a disease in society calculated in terms of early death and disability years [1, 2]. It has been observed that the diagnosis of this disease in the first 90 minutes after the onset of the disease and the initiation of treatment provides benefit for healing [3]. However, the diagnosis is made after imaging by MRI (magnetic resonance imaging) or CT (computed tomography) [4] and given that these devices are non-portable and costly devices, it is difficult to start treatment within the specified time frame, and a clear need for a portable, low cost diagnostic device is observed.

The methods used in biomedical field and their properties are presented in Table I. The dielectric properties of the tissues were examined using microwave (RF), and the importance of these tumors in the differentiation of tissues and the detection of tumor tissues were demonstrated [5,6]. In addition to diagnostic technologies, the thermal effect of the high density microwave was also used for surgery and for the destruction of unhealthy tissues [7]. Similar to microwaves, ultrasonic waves were also used for imaging at low powers, and for thermal treatment at high powers [8,9]. However, it was observed that the resolution was low due to scattering in the microwave imaging studies, whereas the contrast between the different tissues required for the identification of the tissues was low despite the good resolution in the imaging studies performed with ultrasound waves [10]. In recent years, various studies have been carried out on the thermoacoustic imaging method, which combines the superior properties of these two methods and uses pressure waves generated by the application of microwaves at regular intervals on the tissue [10-13].

TABLE I
Methods and properties used for biomedical applications [14]

| | Method | | | | | |
|---|---|---|---|---|---|---|
| | | | | | | Microwave |
| | | | Magnetic | Microwave | | Transmitter-Ultrasound |
| Property | Optic | X-ray | Resonance | (RF) | Ultrasound | Receiver |
| Detection | Low | High | High | Medium | High | High |
| potential in | | | | | | |
| scattering | | | | | | |
| and lossy | | | | | | |
| environment | | | | | | |
| Material | High | Medium | High | High | Medium | High |
| detection | | | | | | |
| capability | | | | | | |
| In-touch | No | No | No | No | Yes | No |
| requirement | | | | | | |
| Safety | Yes | Radiation | Yes | Medium | Yes | Yes |
| Cost | Affordable | Expensive | Expensive | Affordable | Affordable | Affordablee |

The potential of thermoacoustic imaging method for microwave (RF) transmitter-ultrasound receiver systems can be understood when the physical material properties of the tissues given in Table II are examined. The energy transmitted at the RF carrier frequency causes approximately the same amount of heat energy to be absorbed in both the brain and the blood bank. Since the heat capacity is close to each other, the corresponding temperature increase is approximately the same (T_{brain}/T_{blood}=0.8). However, the volumetric heat expansion (β) is 2.5-fold for blood compared to the brain (Table II [15, 16]). This allows thermoacoustic method to differentiate blood clumps accumulated in the brain from brain tissues via brain hemorrhage [14] (εR: Relative dielectric constant, a: Electrical conductivity (S/m), p: density (kg/m3), C: Heat capacity (J/kg/° C.), k: Thermal conductivity (W/m/° C.), β: Volumetric thermal expansion (K−1×10⁻⁴)). Setup and safe operation of the RF transmitter system should comply with specific absorption rate (SAR) requirements not exceeding the 8 W/kg limit in accordance with current regulations [17-20].

TABLE II
Physical material properties of tissues [15, 16]

| Frequency | | | | | | | | |
|---|---|---|---|---|---|---|---|---|
| (MHz) | 1800 | | 2450 | | | | | |
| Tissue | ε_{R} | σ | ε_{R} | σ | ρ | C | k | β |
| | | (S/m) | | (S/m) | (kg/m³) | (J/kg/° C.) | (W/m/° C.) | (K⁻¹ × 10⁻⁴) |
| Skull | 15.56 | 0.43 | 15.01 | 0.57 | 1850 | 3100 | 0.37 | 1.97 |
| Brain | 46.1 | 1.71 | 44.8 | 2.1 | 1046 | 3630 | 0.54 | 1.6 |
| Blood | 59.4 | 2.04 | 58.3 | 2.54 | 1050 | 3617 | 0.52 | 4.0 |

It is thought that the ultrasonic wave due to the volume expansion of the blood bank might suffer from frequency shifts due to the scattering and losses experienced in different environments (brain-skull-air) [14]. Despite these frequency shifts, the airborne ultrasound receiver, capacitive micromachined ultrasonic transducer (CMUT), must have a sufficiently high bandwidth to detect the wave. In order to increase the bandwidth, the squeeze film damping effect was utilized by opening air holes on the membrane [21]. In the CMUT designs examined in the literature, the effects of the presence of air ventilation holes on the membrane or substrate for the vibrating membrane in the conventional mode (no collapse) on bandwidth and CMUT sensitivity (nm/Pa) were examined by theoretical [22] or finite element analysis [23]. The rate of change of the CMUT capacitance and electromechanical coupling efficiency are much higher in the collapse mode than in the conventional mode [24]. It is anticipated that collapse mode operation of airborne CMUT as a receiver has the potential to improve both bandwidth and sensitivity. Collapse mode of the CMUT suffers from charging of insulation layer when the membrane contacts the substrate during operation. To reduce charging of the insulation layer, patterning of electrodes, patterning of insulation layers to reduce the contact area to suppress the effect of charging were proposed in literature [25]. Pre-collapsed membrane configurations at zero DC bias voltage were also proposed to acquire the benefits of the collapse operation [26]. But the fundamental problem is not addressed so far: presence of insulation layer under an electric field at collapse operation causes potentially time-varying charging problems, which changes sensitivity and limits the reliability of the transducer for highly sensitive receiver applications. For CMUTs, which are usually operated in the air, large bandwidth is not generally required. This is due to the fact that the ultrasound waves transmitted at the transmitter frequency of the CMUT are again detected by CMUT at the same frequency [27]. However, to detect blood accumulation in the brain, the ultrasound waves will be generated by RF power delivered to human head at a carrier frequency of 1.8-2.4 GHz and a pulse modulation frequency of 50-300 kHz. Considering the frequency shifts due to absorption, attenuation and scattering of RF-induced ultrasound wave in an environment of greatly varying acoustic impedance of blood, brain, skull and air, the bandwidth and the sensitivity of the airborne ultrasound receiver should be sufficiently large to capture ultrasound waves.

Multi-frequency and multi-band (hyperspectral) imaging techniques are an evolving method for capturing features or traces that conventional single methods cannot. In light-based applications, imaging at wider intervals than the visible spectrum is known to be successful in many areas, such as the detection of qualified agricultural products [28], colorless chemicals [29], oil spills [30] and surface mines [31]. High resolution imaging is also performed by intravascular methods in ultrasound [32].

### SUMMARY

A new method of detecting brain haemorrhage is presented in this document. Our invention is a MEMS airborne ultrasonic transducer system to detect thermoacoustic generation of ultrasound wave caused by the RF-induced volumetric expansion of blood in the brain. At the heart of ultrasonic transducer having higher sensitivity and higher bandwidth is the innovative collapse mode having electrical contact resistance (ECR) feature. Electrical contact resistance force sensing is available in literature [33], however, our invention introduces ECR feature for collapse mode of CMUTs for the first time.

In this invention, detection of blood accumulation in the brain is detected using the thermoacoustic principle. A microwave (RF) carrier frequency (1.8-2.4 GHz) carries energy to the brain within the human safety levels (<8 W/kg) by 50% duty cycle and a pulse modulation frequency of 50-300 kHz. This energy is roughly at the same level as the RF energy emitted by mobile phones. This energy periodically changes the temperature in the tissues in the order of MicroKelvin (10⁻⁶ K) at the modulation frequency. Volumetric thermal expansion (β) of blood is 2.5-fold compared to brain. This difference enables ultrasound waves originating from blood, which is distinguished from the surrounding brain tissue.

In this invention, a novel design of an airborne receiver capacitive micromachined ultrasonic transducer (CMUT) to be operated in collapse mode with electrical contact resistance (ECR) at high sensitivity is also presented to detect very low ultrasound signals. This is achieved by not using any insulation layer and instead using highly resistive dimples to keep reliable collapse operation in our CMUT design. Highly resistive dimples featuring electrical contact resistance are realized by Hertzian contact between poly silicon surfaces (covered with a very thin native oxide of 10 Å enabling tunneling resistance [34]) at collapse operation. Lack of insulation layer solves the common charging problem associated with insulators in high electric field. To obtain this sensitivity, advantages of this novel insulator-free, high-resistance (>10 kΩ) ECR version of collapse mode operation of CMUT is utilized in MEMS ultrasonic receiver. In addition to being ultra-sensitive for detecting cerebral haemorrhage, the detector supports hyperspectral imaging and enhanced bandwidth modes by changing the DC voltage during operational use. Collapse mode of our CMUT offers a wide adjustment range of the central frequency by changing the resistance of the contacting surface via DC bias voltage. Therefore, DC bias-controllable, frequency dependent high sensitivity for the receiver airborne CMUT operating in collapse mode is achieved. Our invention of fast and affordable method for detecting brain haemorrhage based on thermoacoustic principle and airborne resistive-collapse mode CMUT design featuring electrical contact resistance (ECR) serves the ultimate goal of protection of the health and welfare of society.

One aspect of the invention, wherein an RF transmitter and ultrasound receiver systems are combined to transmit RF energy and receive ultrasound wave.

Another aspect of the invention, wherein RF transmitter system includes an RF signal generator, RF amplifier and horn antenna.

Another aspect of the invention, wherein ultrasound receiver system includes a lock-in amplifier, a DC supply, two ultrasonic transducer arrays wirebonded to low noise amplifier (LNA) chips.

Another aspect of the invention, wherein the ultrasonic transducer array is composed of independent four transducers in 2×2 CMUT configuration.

Another aspect of the invention, wherein four transducers in the array differ in membrane size to have incremental difference in resonance frequency from one another.

Another aspect of the invention, wherein the transducer is airborne capacitive micromachined ultrasonic transducer (CMUT).

Another aspect of the invention, wherein the transducer is not touching or making contact, i.e., operating freely in air.

Another aspect of the invention, wherein the transducer has poly silicon membrane having poly silicon dimples facing the bottom electrode.

Another aspect of the invention, wherein the diameter of dimples is set to 8 μm.

Another aspect of the invention, wherein the transducer has poly silicon bottom electrode.

Another aspect of the invention, wherein the top and bottom poly silicon electrodes are covered by very thin native oxide (10 Å) enabling tunneling resistance.

Another aspect of the invention, wherein there is no insulation layer keeping top and bottom electrodes from passing current in-between at membrane collapse.

Another aspect of the invention, wherein the dimples are spatially distributed on the contacting surface of the membrane.

Another aspect of the invention, wherein dimples form Hertzian contact with the substrate at membrane collapse.

Another aspect of the invention, wherein electrical contact resistance (ECR) is observed at Hertzian contact of the dimples.

Another aspect of the invention, wherein the control range of the membrane against ultrasound stimulation and the sensitivity of the measuring system are adjusted by controlling the DC bias voltage after membrane collapse.

Another aspect of the invention, wherein the DC bias voltage can be changed down to snapback voltage or changed up beyond the collapse voltage.

Another aspect of the invention, wherein the transducer operated in resistive-collapse (R-collapse) mode, i.e., collapse mode with electrical contact resistance (ECR).

Another aspect of the invention, wherein specifications of ultrasonic transducer are, Collapse voltage is 1.4 V.

Snapback voltage is 1.25 V.

Impedance model parameters R_{S}, C_{S} and R_{P} are 150 Ω, 36.7 pF and 15.2 kΩ, respectively.

Another aspect of the invention, wherein the transducer operates reliably at resistive-collapse mode.

Another aspect of the invention, wherein the transducer is wirebonded to LNA chip.

Another aspect of the invention, wherein RF signal generator generates a pulse modulated RF carrier signal.

Another aspect of the invention, wherein the RF signal generator is connected to lock-in amplifier.

Another aspect of the invention, wherein the RF signal generator sweeps the pulse modulation frequency from 50 kHz up to 300 kHz.

Another aspect of the invention, wherein the DC bias of transducer array is adjusted for maximum sensitivity for the present pulse modulation frequency.

Another aspect of the invention, wherein the lock-in amplifier tracks the pulse modulation frequency.

Another aspect of the invention, wherein the lock-in amplifier measures the signal coming from LNA to calculate the spectral ultrasound power at a certain frequency for a specific blood size to benefit from constructive and destructive interference of RF-induced blood-originating ultrasound waves.

Another aspect of the invention, wherein the lock-in amplifier uses time-gated mode to process only a certain time waveform interval between t_{START} and t_{STOP} (referenced to trigger signal from the RF signal generator) determined from ultrasound time-of-flight calculation for a certain region within the brain.

Another aspect of the invention, wherein lock-in amplifier data collected from #1 MEMS ultrasonic transducer and #2 MEMS ultrasonic transducer, each having 4 units (CMUT #1 to CMUT #4), are processed with multi-frequency and multi-band (hyperspectral) imaging techniques.

Another aspect of the invention, wherein all instruments are controlled by a personal computer and a software.

### BRIEF DESCRIPTION OF THE DRAWINGS

The figures used to better explain MEMS airborne ultrasonic transducer system developed with this invention and their descriptions are as follows:

FIGS. 1A and 1B Principle of operation for RF transmitter-ultrasound receiver system for detection of blood bank in brain

FIG. 1A RF transmission towards tissue having skull, brain and blood.

FIG. 1B Ultrasound receiver picking up ultrasound wave due to thermoacoustic expansion of tissue at the RF modulation frequency.

FIG. 2 Axisymmetrix finite element model (PZFlex software) to determine the ultrasound wave generated due to thermoacoustic expansion of tissue.

FIGS. 3A and 3B Finite element simulation results of ultrasound wave generated by blood bank (hₐᵢᵣ=1 cm, hₛₖ=0.7 cm, h_{br}=20, h_{bl}=1 cm, r_{bl}=1 cm).

FIG. 3A 100 kHz single pulse triangular wave with a temperature of 1 C increasing (0 μs-5 μs) and decreasing (5 μs-10 μs) of the blood bank due to the expansion of ultrasonic wave caused by expansion at the time of t=15 μs.

FIG. 3B Reflection of the same ultrasonic wave by the skull at t=80 is.

FIGS. 4A-4D Time domain simulation results of pressure with burst cycle of 10.

FIG. 4A Pressure time waveform when brain having blood bank was simulated.

FIG. 4B Pressure time waveform when brain without any blood bank was simulated.

FIG. 4C Pressure time waveform for difference of time waveforms in FIG. 4A and FIG. 4B.

FIG. 4D Pressure time waveform when only blood (not brain) was assumed to be expanding due to RF energy transfer.

FIGS. 5A and 5B Fast Fourier Transform (FFT) of pressure time waveform.

FIG. 5A FFT of pressure time waveform in FIG. 4C.

FIG. 5B FFT of pressure time waveform in FIG. 4D.

FIGS. 6A-6C Time domain simulation results for pressure at different modulation frequency FIG. 6A Modulation frequency of 100 kHz generating a peak pressure of 45 Pa/K.

FIG. 6B Modulation frequency of 150 kHz generating a peak pressure of 24 Pa/K.

FIG. 6C Modulation frequency of 225 kHz generating a peak pressure of 104 Pa/K.

FIG. 7 MEMS airborne ultrasonic transducer system setup to detect thermoacoustic generation of ultrasound wave caused by the RF-induced volumetric expansion of blood in the brain.

FIG. 8 Schematic drawing of MEMS ultrasonic transducer array (2×2 CMUT) placed on a low noise amplifier (LNA) chip.

FIGS. 9A and 9B Design and microfabrication of MEMS ultrasonic transducer array.

FIG. 9A Mask layout design (Tanner Tools software) for MEMS ultrasonic transducer array (2×2 CMUT).

FIG. 9B Microscope image of actual microfabricated MEMS ultrasonic transducer array (2×2 CMUT) with electrical pads for wirebond.

FIG. 10 Cross-sectional view of the MEMS ultrasonic transducer design.

FIGS. 11A and 11B Hole and dimple arrangement for the membrane.

FIG. 11A Schematic drawing of hole and dimple arrangement on the membrane.

FIG. 11B Microscope image showing hole and dimple arrangement of the actual microfabricated membrane in the second quadrant.

FIGS. 12A and 12B Input impedance representation for CMUT.

FIG. 12A Input impedance representation for CMUTs in conventional (no contact between the membrane and the substrate) and collapse (having an insulation layer between the membrane and the substrate preventing DC current flow) mode.

FIG. 12B Input impedance representation for our novel CMUT design featuring resistive dimples, i.e., electrical contact resistance (ECR), to limit current flow in collapse mode. There is no insulation layer between the membrane and the substrate.

FIG. 13 Laser vibrometer measurement setup FIGS. 14A and 14B Laser vibrometer displacement measurements of MEMS ultrasonic transducer showing collapse and snapback behavior.

FIG. 14A Displacement of center position (at a radial distance of 13 μm) of MEMS membrane in conventional and collapse mode operation.

FIG. 14B Displacement of radial middle point (at a radial distance of 96 μm) of MEMS membrane in conventional and collapse modes.

FIGS. 15A and 15B Laser vibrometer displacement measurements of MEMS ultrasonic transducer.

FIG. 15A Displacement of MEMS membrane as a function of radial position under conventional (V_{DC}=[0.75 V, 1 V, 1.25 V], f=[45 kHz, 40 kHz, 40 kHz]) and collapse (V_{DC}=[1.5 V, 1.75 V], f=[135 kHz, 140 kHz]) modes.

FIG. 15B Average displacement of MEMS membrane as a function of frequency under conventional (V_{DC}=[0.75 V, 1 V, 1.25 V]) and collapse (V_{DC}=[1.5 V, 1.75 V]) modes.

FIGS. 16A and 16B Laser vibrometer displacement measurements of MEMS ultrasonic transducer.

FIG. 16A Displacement of MEMS membrane operating in conventional mode (V_{DC}=1.25 V) as a function of radial position and frequency.

FIG. 16B Displacement of MEMS membrane operating in collapse mode (V_{DC}=1.75 V) as a function of radial position and frequency.

FIGS. 17A and 17B Impedance characterization of MEMS ultrasonic transducer.

FIG. 17A Series capacitance of MEMS ultrasonic transducer in conventional (V_{DC}=[0 V]) and collapse (V_{DC}=[1.5 V, 1.75 V, 2 V]) modes.

FIG. 17B Series resistance of MEMS ultrasonic transducer in conventional (V_{DC}=[0 V]) and collapse (V_{DC}=[1.5 V, 1.75 V, 2 V]) modes.

### DETAILED DESCRIPTION OF THE EMBODIMENTS

The present invention has been described in detail in the following. This invention offers a new method of detecting brain haemorrhage.

In this section, a novelty is going to be demonstrated.

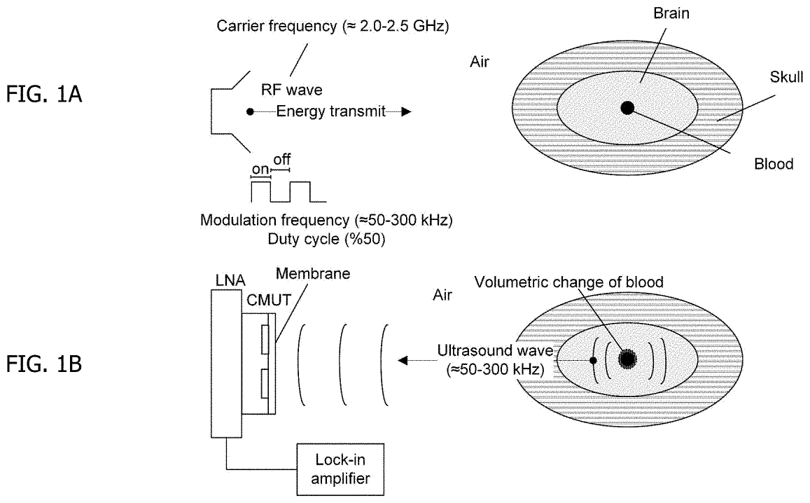

Our invention is a MEMS airborne ultrasonic transducer system to detect thermoacoustic generation of ultrasound wave caused by the RF-induced volumetric expansion of blood in the brain (FIGS. 1A-1B). An RF signal with an on/off modulation frequency between 50-300 kHz carries energy to the brain within the human safety levels (<8 W/kg) [35]. This energy periodically changes the temperature in the tissues in the order of μK at the modulation frequency. Volumetric thermal expansion coefficient (β) of blood is 2.5-fold compared to that of brain. This difference enables detection of blood accumulation of certain size, i.e., blood-originated ultrasound waves from the surrounding brain tissue are detected in spite of the high attenuation of skull bone surrounding the brain. It is known that 1 mK temperature increase generates 800 Pa pressure on the source [36]. This pressure due to thermal expansion is calculated by equation (1), where p(r,t) (Pa) is the pressure occurring at time t at a position r (m), v (m/s) is the velocity of the ultrasound wave, β (1/K) is the thermal expansion coefficient, C (J/kg·K) is the specific heat capacity, and Q(J) is the thermal energy absorbed by the brain.

$$( {▽}^{2} - \frac{1}{{v}^{2}} \frac{\partial }{\partial {t}^{2}} ) p ( r , t ) = - \frac{β}{C} \frac{\partial Q ( r , t )}{\partial t} \qquad ( 1 )$$

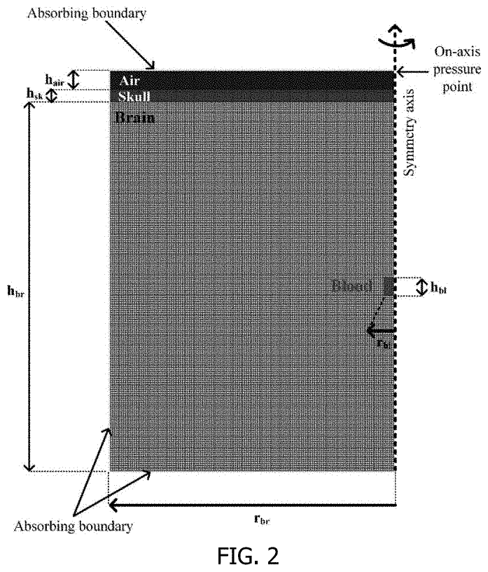

Our axisymmetric 2D finite element model shown in FIG. 2 was composed of minute amount of blood (h_{bl}=1 cm, r_{bl}=1 cm) within the brain tissue (h_{br}=20 cm, r_{br}=11 cm) surrounded by the skull bone (hₛₖ=0.7 cm). This model is a viable representation of a typical human head for our purpose. The material properties for thermoacoustic finite element analysis are given in Table III. The pressure within the air (hₐᵢᵣ=1 cm) above the skull bone was calculated. In this model, attenuation of the skull was properly modeled with linearly frequency dependent attenuation parameters of 20 dB/cm and 60 dB/cm at 1 MHz for longitudinal and shear waves, respectively.

TABLE III
Material properties for simulation.

| | Material Properties | | | |
|---|---|---|---|---|
| Material | p | β | Longitudinal | Shear |
| Type | (kg/m³) | (K⁻¹ × 10⁻⁴) | velocity (m/s) | velocity (m/s) |
| Skull bone | 1850 | 1.97 | 3400 | 1760 |
| Brain | 1046 | 1.60 | 1560 | — |
| Blood | 1050 | 4.00 | 1500 | — |
| Air | 1 | — | 340 | — |

The finite element analysis was performed using double precision solver of a commercially available software package (PZFlex). On-axis pressure point shown in FIG. 2 was used to collect pressure waveform. This point was on the symmetry axis aligned with the blood clot. We assumed the surrounding air had a thickness of 1 cm, and skull bone had a thickness of 0.7 cm. Absorbing boundary conditions were properly set in the model (FIG. 2).

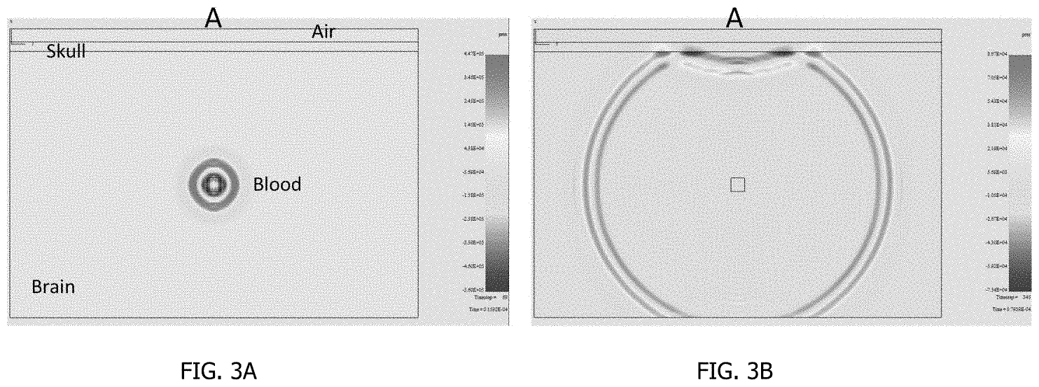

Initially, 100 kHz single pulse triangular wave with a temperature of 1 C increasing (0 μs-5 μs) and decreasing (5 μs-10 μs) of the blood bank (no thermal expansion of brain) was applied and ultrasonic wave caused by thermal expansion at the time of t=15 μs was observed in FIG. 3A. Reflection of the same ultrasonic wave by the skull at t=80 μs was observed in FIG. 3B. Thermal expansion coefficients of blood, brain and skull bone were used in the thermal analysis. A temperature increase in tissues was applied for 10 cycles as a triangular waveform at an ultrasonic modulation frequency, which launched ultrasonic waves as a result of volumetric expansion of blood. Due to the nature of RF heating, temperature increase in blood will be accompanied by similar changes in the temperatures of brain and skull bone. Assuming uniform electric field within the brain, the temperature changes in brain and skull bone will be approximately proportional with their conductivities. Under these assumptions, RF heating in the tissues will result in approximate temperature increases proportional to 1 K, 0.8 K and 0.2 K for blood, brain and skull bone; respectively.

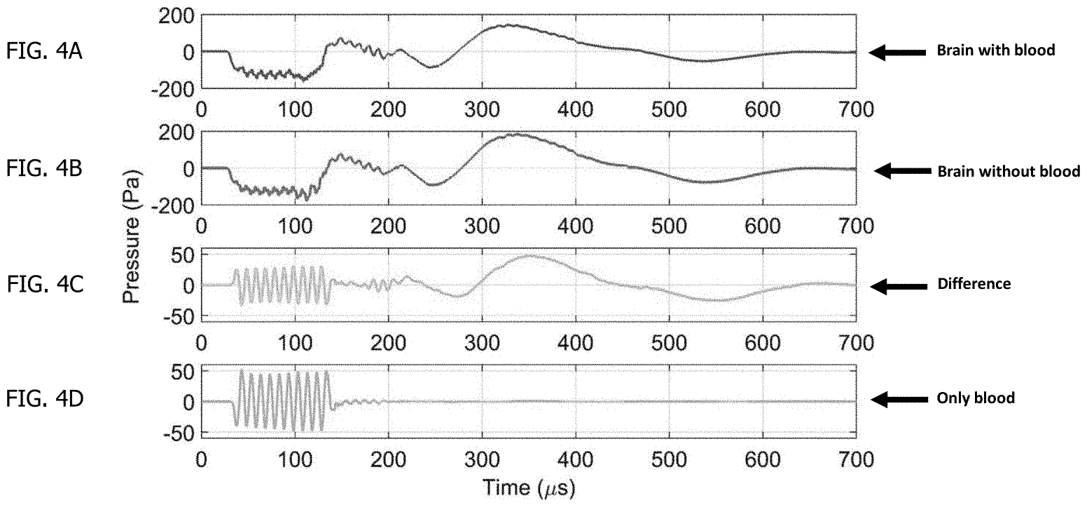

For an RF signal with an on/off modulation frequency of 100 kHz, time domain finite element simulations were performed under the assumption of brain, blood and skull bone being simultaneously heated with a 10 cycle triangular waveform. The pressure waveforms are shown in FIGS. 4A-4D. The pressure for the case of brain having blood at a distance of 1 cm away from the skull bone is given in FIG. 4A, whereas the pressure for the case of brain without any blood is given in FIG. 4B. The difference of these waveforms representing the effect of blood bank was extracted and shown in FIG. 4C. If thermal expansions of brain and skull bone were neglected in the FEA, the pressure due to expansion of only blood was calculated as in FIG. 4D.

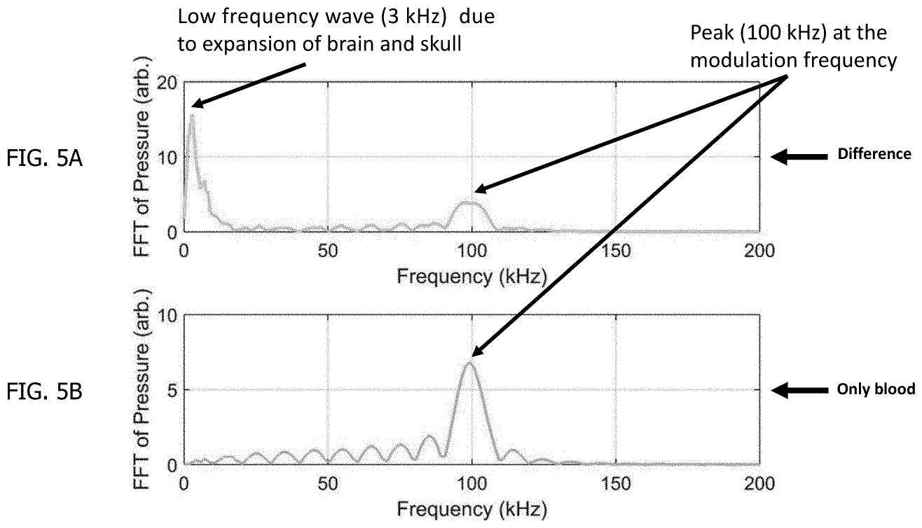

Fast Fourier Transforms (FFT) of pressure waveforms in FIG. 4C and FIG. 4D were performed on the full data without any filtering, and the FFT results are shown in FIG. 5A and FIG. 5B, respectively. Both curves presented a peak at 100 kHz matching to the modulation frequency. However, expansion of brain and skull had an additional peak around 3 kHz representing a variation at a significantly lower frequency.

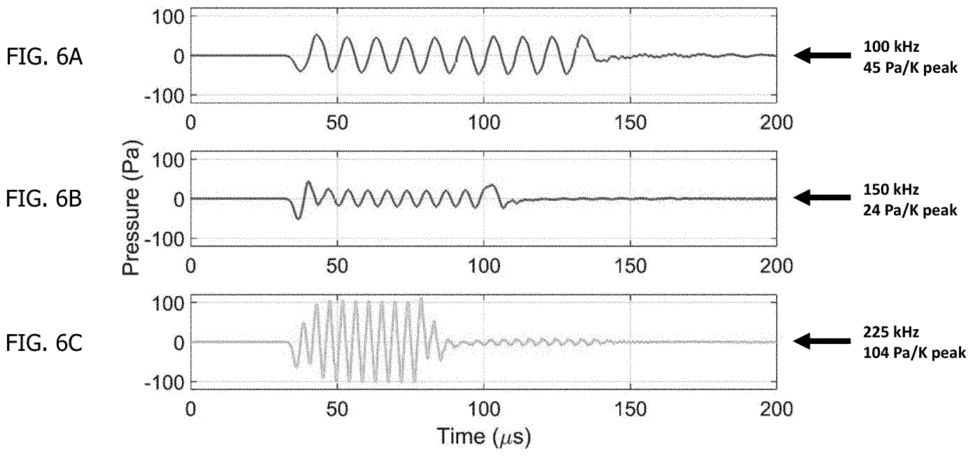

Pressure waveform in FIG. 4D acquired at 100 kHz was redrawn as in FIG. 6A to act as the reference signal (45 Pa/K peak pressure) for exploring the effect of modulation frequency. For a modulation frequency of 150 kHz, the signal peak is reduced to 24 Pa/K due to destructive interference of waves launched from the finite-sized (h_{bl}=1 cm) blood clot (FIG. 6B). For the same blood clot, using a modulation frequency of 225 kHz, the signal peak is boosted up to 104 Pa/K due to constructive interference (FIG. 6C). Modulation frequency for constructive or destructive interference provides us the information about the size of blood clot in the brain compared to wavelength of ultrasound wave in blood medium [36].

Specific absorption rate (SAR) is defined in equation (2). Based on theoretical calculations described in equations (2) and (3), RF heat delivered to the tissue can be related to accompanying increase in its temperature (ΔT). Uniform electric field, E(r), is assumed within the head, and using the material properties in Table IV, normalized temperature increase ratios for brain and skull bone with respect to blood are calculated to be 0.83 and 0.24, respectively.

$$SAR = \frac{1}{Volume} {\int }_{sample} \frac{σ ( r ) {\vert E ( r ) \vert}^{2}}{ρ ( r )} dr \qquad ( 2 )$$

$$SAR \times \frac{Duty Cycle}{Frequency} = C \times Δ T \qquad ( 3 )$$

The

TABLE IV
Material properties for theoretical calculations

| | Material properties | | | |
|---|---|---|---|---|
| | | | | ΔT/ΔT_{bl} |
| Material | Density, ρ | Conductivity, σ | Heat Capacity, C | α |
| type | (kg/m³) | (S/m) | (J/kg × K) | (σ/C × ρ) |
| Skull | 1850 | 0.43 | 3100 | 0.24 |
| bone(sk) | | | | |
| Brain (br) | 1046 | 1.71 | 3630 | 0.83 |
| Blood (bl) | 1050 | 2.04 | 3617 | 1 |

finite element simulation results are summarized in Table V. Using the maximum allowed average heat power of 8 W/kg at a duty cycle of 50% in equation (3), temperature increase in the blood over a cycle was calculated to be in the range of μK as given in Table V. Considering the minimum detectable pressure level of approximately 0.9 mPa for a CMUT receiver in air [38, 39], signal-to-noise ratio (SNR) should be increased by averaging techniques [40]. This technique for collecting data will improve the SNR with the square root of the number of samples [40, 41].

TABLE V
Summary of finite element simulation results

| | FEA # | | |
|---|---|---|---|
| Property | 1 | 2 | 3 |
| Frequency (kHz) | 100 | 150 | 225 |
| Blood dimension (cm) | 1 | 1 | 1 |
| Depth (cm) | 1 | 1 | 1 |
| Temperature increase (μK) | 0.022 | 0.015 | 0.010 |
| Temperature dependent pressure (Pa/K) | 45 | 25 | 104 |
| Pressure (μPa) | 0.99 | 0.38 | 1.04 |
| Burst frequency (kHz) | 5 | 5 | 5 |
| Data collection time (min) | 2.8 | 19.1 | 2.5 |

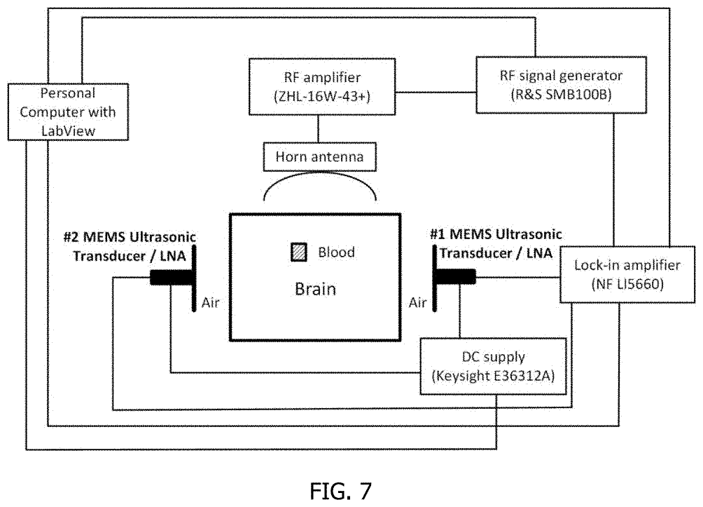

MEMS airborne ultrasonic transducer system setup proposed to detect thermoacoustic generation of ultrasound wave caused by the RF-induced volumetric expansion of blood in the brain is given in FIG. 7. This proposed setup includes RF signal generator (SMB100B, Rohde&Schwarz), RF amplifier (ZHL-16 W-43+, Minicircuits) and a horn antenna as part of the RF transmitter part. The horn antenna will be placed slightly above the head denoted as brain in FIG. 7. This placement will expose the whole brain to RF energy during transmission. The proposed setup includes lock-in amplifier (LI5660, NF), DC supply (E36312A, Keysight) and 2 identical units of MEMS ultrasonic transducer electrically connected to low noise amplifier (LNA) chip (MAX4805, Maximintegrated) as part of the ultrasound receiver part. RF signal generator is connected to lock-in amplifier for trigger synchronization. Lock-in amplifier with a dynamic reserve of more than 100 dB will collect and average data while sweeping frequency (locked to modulation frequency of the RF signal generator) with a very small bandwidth (mHz) suppressing noise and achieving high signal-to-noise ratio (SNR). Personal computer with a control software (LabView) manages the RF signal generator, the lock-in amplifier and the DC supply. MEMS ultrasonic transducer is placed roughly 1-cm away from the head, and does not touch the head. Hence, it operates in air. Airborne MEMS ultrasonic transducer, a capacitive micromachined ultrasonic transducer (CMUT), is a novel aspect of this invention in that it operates in Resistive-collapse (R-collapse) mode, i.e., collapse mode with electrical contact resistance (ECR), for the first time.

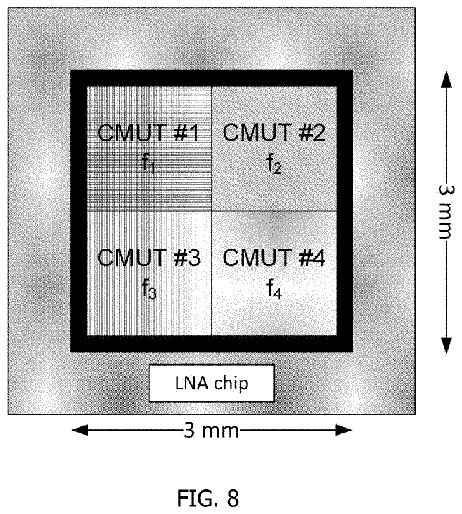

MEMS ultrasonic transducer array (2×2 CMUT) placed on a low noise amplifier (LNA) chip is schematically shown in FIG. 8. CMUT #1 to CMUT #4 have the same dimensions except the membrane diameter gradually changing to have varying center frequency for the purpose of enabling hyperspectral analysis. CMUT #1 to CMUT #4 are electrically isolated from each other, and have a separate amplifier module for each from the LNA chip.

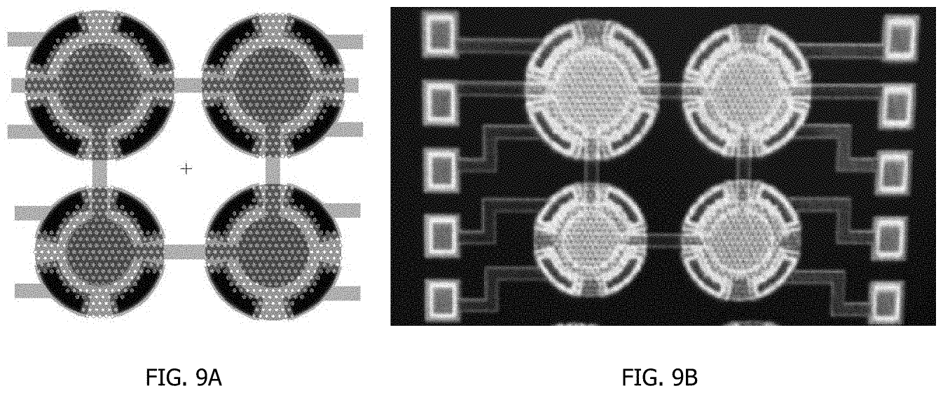

Mask design and actual realization of MEMS ultrasonic transducer array are given in FIGS. 9A and 9B. Mask layout design (Tanner Tools software) for MEMS ultrasonic transducer array (2×2 CMUT) is shown in FIG. 9A. The masks were designed for a commercially available foundry service (Polymumps, MEMSCAP). Based on commercially available multi-user multi-processes (MUMPS) offered by foundries, POLYMUMPS process (MEMSCAP) was selected due to its suitability for microfabrication of airborne membranes supported by the non-limiting process design rules for our intended application. Furthermore, reproducibility and consistency of this mature process is considered to be advantageous for the realization of high fidelity membrane. Microscope image of actual microfabricated MEMS ultrasonic transducer array (2×2 CMUT) with electrical pads for wirebond is shown in FIG. 9B.

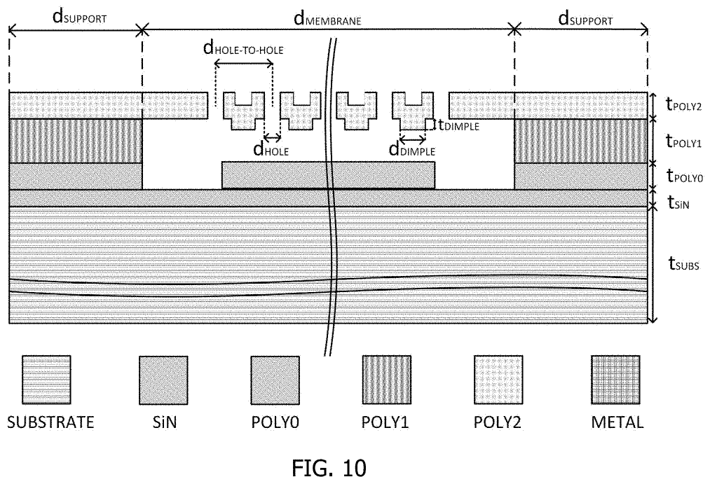

Cross-sectional view of the MEMS ultrasonic transducer design is schematically given in FIG. 10. The dimensions are given in Table VI.

TABLE VI
Values of the representative dimensions of the design.

| Dimension parameter | Value |
|---|---|
| | #1: 500 |
| Membrane diameter (d_{MEMBRANE}), μm | #2: 470 |
| | #3: 440 |
| | #4: 410 |
| Support length (d_{SUPPORT}), μm | 50 |
| Hole-to-hole diameter (d_{HOLE-To-HOLE}), μm | 28 |
| Dimple diameter (d_{DIMPLE}), μm | 8 |
| Hole diameter (d_{HOLE}), um | 16 |
| Metal thickness (t_{METAL}), um | No metal |
| | on |
| | membrane, |
| | 0.5 on |
| | pads |
| POLY2 thickness (t_{POLY2}), μm | 1.5 |
| Dimple thickness (t_{DIMPLE}), μm | 0.75 |
| POLY1 thickness (t_{POLY1}), μm | 2.0 |
| POLY0 thickness (t_{POLY0}), μm | 0.5 |
| SiN thickness (t_{SiN}), μm | 0.6 |
| Substrate thickness (t_{SUBS}), μm | >650 |

This process is based on polysilicon layers. The ability to design membranes and the ability to etch sacrificial oxide layers under the polysilicon layers makes this process valuable for our design. Obtain perfect etching of sacrificial oxide layers requires placement of holes in the polysilicon layers. The distance between any etching holes cannot be larger than 30 μm. CO₂ dry etch in addition to the standard HF wet etch for oxide removal was used. CO₂ dry was used to prevent the stiction of the adhesion between the membrane and the substrate for the large aspect ratio used in the membrane (1:200). Very low compressive stress (<7 MPa) of POLY2 membrane material with a thickness of 1.5 μm made our large aspect-ratio membrane having negligible curvature due to residual stress.

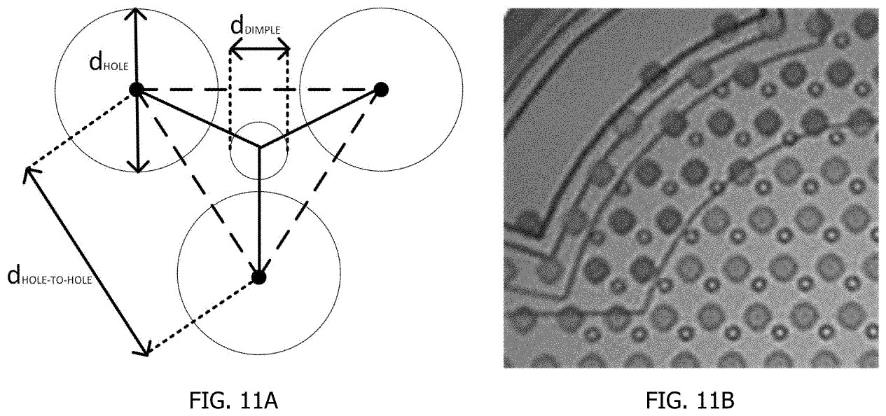

Important things to note in this design are
There is no metal deposition on the membrane (FIG. 10). POLY2 membrane having a sheet resistance of 20 ohm/square (resistivity of 3×10⁻³ ohm-cm) acts as the conductor for the top electrode. Metal deposition is only done on pads for the purpose of wirebond (Table VI).
Dimple diameter is selected to be a small value, 8 μm, so that once the top electrode of POLY2 collapses onto bottom electrode of POLY0 having a sheet resistance of 30 ohm/square (resistivity of 1.5×10⁻³ ohm-cm), current flow can be limited by the relatively large resistance due to smaller contact area. Actual contact diameter will be in fact even smaller due to curvature of the dimple surface caused my microfabrication. Contact is of standard Hertzian contact type, which has the maximum mechanical pressure on the center of the dimple and electrical current density will be maximum on the rim of the contacting surface [42]. It is important to note that small dimple diameter and curvature of the dimple surface acting as a Hertzian contact limits the contacting surface area. In addition to this, low electrical resistivity of POLY2 and POLY0 contacting surfaces further constricts the electrical current flow to mainly the rim of the dimple contact surface. Furthermore, a native oxide of 10 Å on both poly silicon surfaces enable tunneling resistance. Therefore, a highly resistive dimple contact resistance, i.e, electrical contact resistance or tunnel resistance [34], is formed.
There is no insulation layer protecting top electrode and bottom electrode to short circuit at collapse. Current flow at collapse is limited by the high resistance presented by polysilicon layers forming top electrode, dimple and bottom electrode. Therefore, successful collapse operation without electrical failure due to lack of insulation layer is achieved thanks to high electrical resistance between the electrodes at membrane collapse.
Lack of insulation layer removes the charging problem observed at collapse operation.
Dimple is placed at the center of gravity of every other triangle formed by neighboring holes (FIG. 11A, FIG. 11B).
Fill factor of the membrane is approximately 70%, meaning that 30% of the membrane is covered by holes. For an airborne transducer with higher receive sensitivity to ultrasound, percentage of holes should be reduced to less than 1% [21]. For our design based on POLYMUMPS process, coverage of the holes to satisfy this requirement can be done with Parylene coating [43]. Effect of such coating changes the resonance frequency of the membrane by covering the holes, but the main features of resistive collapse mode is unaffected and holds true.

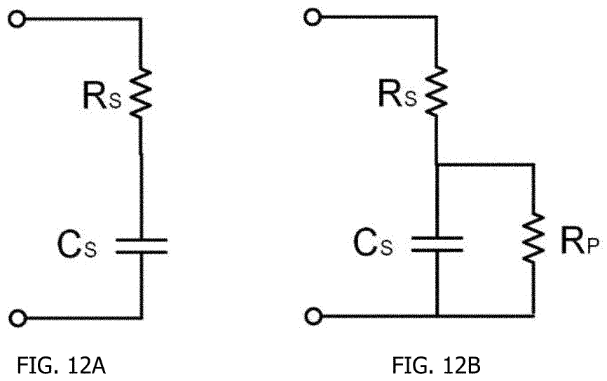

Input impedance representation for CMUTs in conventional (no contact between the membrane and the substrate) and collapse (having an insulation layer between the membrane and the substrate preventing DC current flow) mode is given in FIG. 12A. Serial connection of resistance (R_{S}) and capacitance (C_{S}) represents the input impedance of the CMUT.

Input impedance representation for our novel CMUT design featuring highly resistive dimples to form current flow in collapse mode is given in FIG. 12B. There is no insulation layer between the membrane and the substrate. Therefore, at collapse mode, a resistance (R_{P}) in parallel with capacitance (C_{S}) is added, and hence, named as Resistive-collapse (R-collapse) mode. Input impedance representation of FIG. 12B can be converted to that of FIG. 12A using serial connection of resistance (R_{S-equ}) and capacitance (C_{S-equ}) as shown in equations (4) and (5).

$${C}_{S - equ} = {C}_{S} + \frac{1}{{w}^{2} {C}_{S} {R}_{P}^{2}} \qquad ( 4 )$$

$${R}_{S - equ} = {R}_{S} + \frac{{R}_{P}}{1 + {w}^{2} {C}_{S}^{2} {R}_{P}^{2}} \qquad ( 5 )$$

R-collapse mode enables important features (dependency on frequency (w: angular frequency in rad/s, f=w/2π in Hz) and dimple resistance (R_{P})) as a novelty to be explored in our invention.

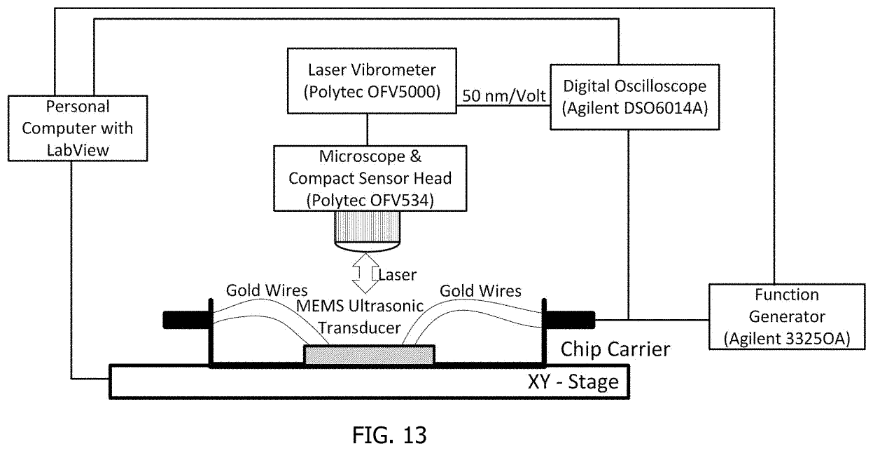

In general, an insulation layer is needed to prevent top and bottom electrodes to short circuit when membrane collapses onto the substrate. Membrane and substrate surfaces will touch and form a flat mechanical contact region having an electrical conductive path. In our design, first we selected both contacting surfaces made of polysilicon having high resistivity compared to metals roughly differing by 5 orders of magnitude. Second, right underneath the membrane, our design had dimples of small diameter and curved structure to form small-sized hertzian contact at membrane collapse. Third, placement of dimples at every other geocentric center of hole triangles (FIG. 11A, FIG. 11B) provide reduction of dimple resistance (R_{P}) in FIG. 12B as DC bias voltage is increased even after collapse. These features enable advancement of receive sensitivity for a MEMS ultrasonic transducer operating in resistive-collapse (R-collapse) mode. Laser Vibrometer (OFV5000/OFV534, Polytec) is used together with the digital oscilloscope (DS06014A, Agilent), the function generator (33250A, Agilent) and a personal computer with LabView (National Instruments) on it to control the devices in the setup (FIG. 13). Characterization via laser vibrometer is based on the detection of the displacement of the MEMS membrane as a result of the electrical excitation. The velocity decoder (VD-09, Polytec) with range selection of 20 mm/s/V providing a high frequency cutoff of 1 MHz was used in measurements due to its low frequency operational capacity. A laser light at 633 nm wavelength is sent to the membrane and the reflected light is used to understand the deflection of the membrane via the interferometer that is utilized between the membrane and the laser light. Besides the general response of the membrane, this characterization setup enables the spatial displacement inspection of the whole membrane. In other words, by directing the laser light on different points on the membrane, spatial displacement response of the membrane to any excitation can also be obtained.

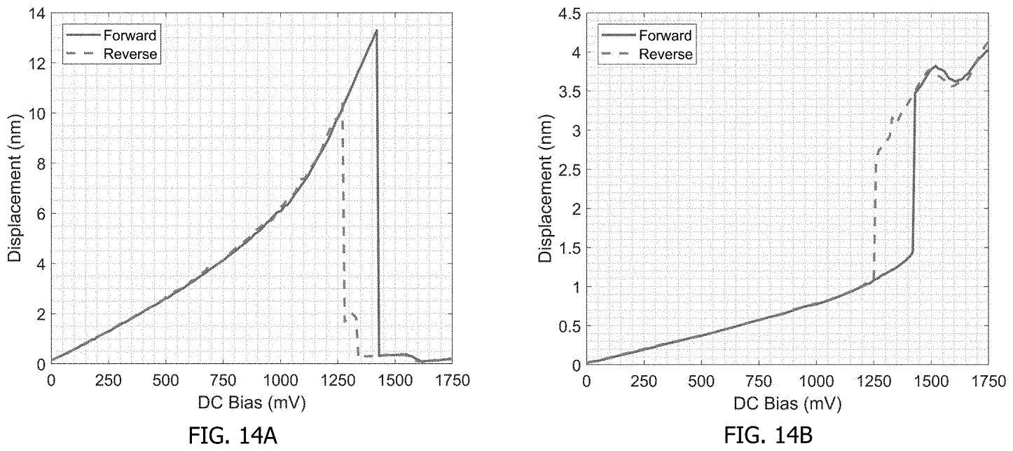

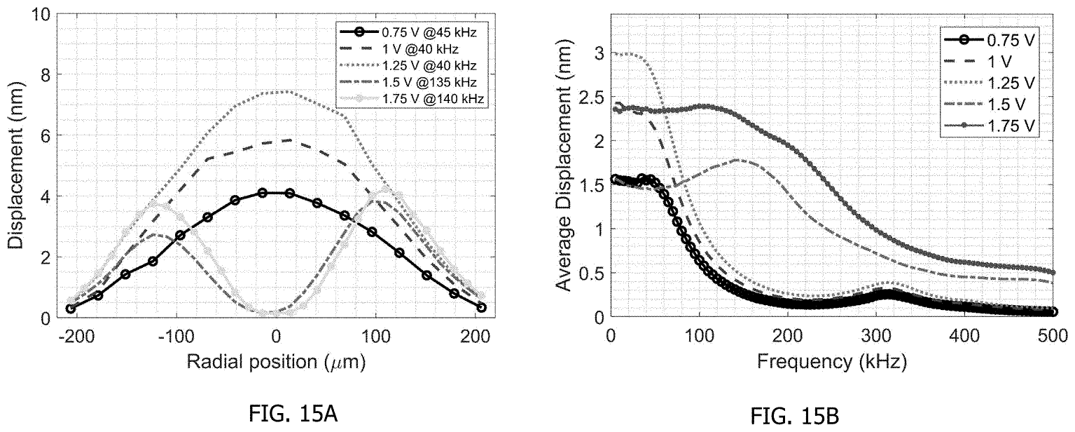

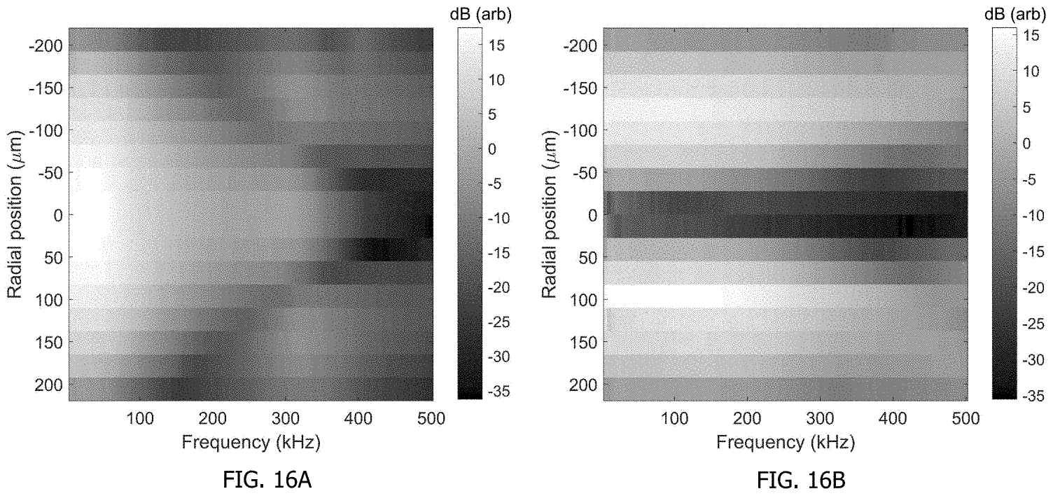

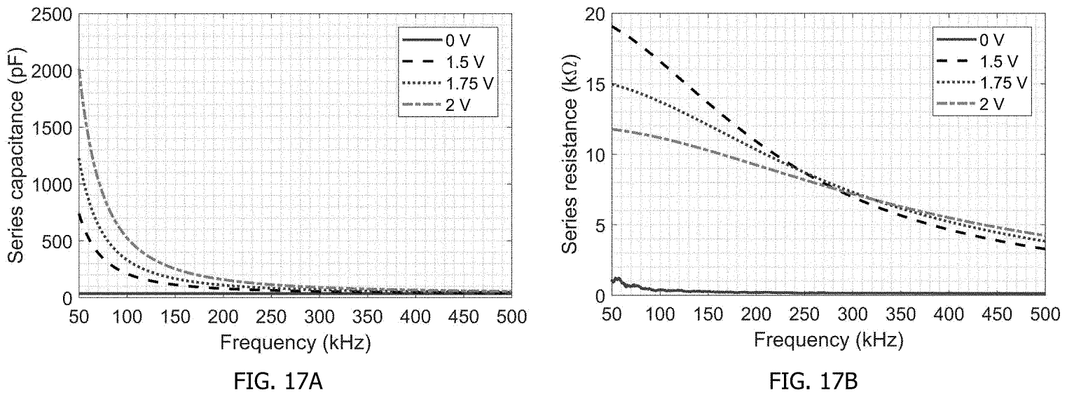

MEMS ultrasonic transducer, CMUT #3 having a membrane diameter of 440 μm (Table VI), was characterized via laser vibrometer. Other CMUTs (#1, #2, #4) will be similar to CMUT #3 with varying resonance frequency (also, collapse and snapback voltages) due to changes in membrane diameter. Laser vibrometer displacement measurements of MEMS ultrasonic transducer showing collapse and snapback behavior is shown in FIGS. 14A and 14B. Displacement of center position (at a radial distance of 13 μm) of MEMS membrane in conventional and collapse mode operation is given in FIG. 14A. A continuous wave (CW) AC voltage of 0.1 Vₚ₋ₚ at a frequency of 40 kHz (resonance frequency in the conventional mode of operation) was applied while the DC bias voltage was increased from 0 V up to 1.75 V in the forward data (FIG. 14A). Collapse voltage of the transducer was determined as 1.4 V. Then, the DC bias voltage was decreased from 1.75 V down to 0 V in the reverse data (FIG. 14A). Snapback voltage of the transducer was determined as 1.25 V. Maximum displacement of a membrane is observed at the center position in the conventional mode, whereas after collapse of the membrane, the maximum displacement of the collapsed membrane is observed at a point close to a radial middle point. Also, the resonance frequency shifts towards a higher frequency. Displacement of radial middle point (at a radial distance of 96 μm) of MEMS membrane in conventional and collapse modes are shown in FIG. 14B. A continuous wave (CW) AC voltage of 0.1 Vₚ₋ₚ at a frequency of 140 kHz (resonance frequency in the collapse mode of operation) was applied while the DC bias voltage was swept in the forward and reverse directions. Displacement of MEMS membrane as a function of radial position under conventional and collapse modes is shown in FIG. 15A. AC voltage of 0.1 Vₚ₋ₚ was kept constant whereas the frequency of AC voltage was selected as the resonance frequency (f₀) observed at the DC bias voltage applied. For example, the membrane had an fₒ of 45 kHz at V_{DC}=0.75 V. At V_{DC}=1 V, fₒ decreased to 40 kHz due to spring softening. At V_{DC}=1.5 V, fₒ increased to 135 kHz due to collapse. At V_{DC}=1.75 V, fₒ increased to 140 kHz due to enlarged contact region. Average displacement of MEMS membrane as a function of frequency under conventional and collapse modes is given in FIG. 15B. Displacement of MEMS membrane operating in conventional mode (V_{DC}=1.25 V) as a function of radial position and frequency is given in FIG. 16A. Displacement of MEMS membrane operating in collapse mode (V_{DC}=1.75 V) as a function of radial position and frequency is given in FIG. 16B. Our transducer design operating in R-collapse mode presents higher average sensitivity over a broader bandwidth. To characterize the input impedance of the transducer, network/impedance analyzer (5061B, Keysight) was used. Series capacitance and series resistance values are shown in FIG. 17A and FIG. 17B, respectively. In the conventional mode, these values are fairly constant; series capacitance is almost unchanged around 36 pF as a function of frequency from 50 kHz to 500 kHz at an input power of −10 dBm (FIG. 17A). Because there is no contact between the membrane and the substrate surfaces in conventional mode, R_{P} is infinite; i.e., there is no R_{P} in the impedance model (FIG. 12A). In collapse mode (V_{DC}=1.5 V, V_{DC}=1.75 V, V_{DC}=2 V), these values (resistance (R_{S-equ}) and capacitance (C_{S-equ})) changed showing the expected behavior as derived in equations (4) and (5). Using data available in FIG. 17A and FIG. 17B, using equations (4) and (5), for DC bias voltage of 1.75 V, C_{S}=36.7 pF, R_{S}=150Ω and R_{P}=15.2 kΩ were calculated. From DC bias voltage of 0 V changed to DC bias voltage of 1.75 V, C_{S} increased a fraction of a pF due to collapse, not a drastic change due to dimple thickness of 0.75 μm (Table VI) still keeping the membrane and substrate surfaces away from each other except the small dimple contact area touching mechanically and conducting electrically (FIG. 11B). But, due to contact, dimple contact resistance (R_{P}) came into play, changing impedance model to that shown in FIG. 12B. Conversion of equivalent circuit having R_{S}, C_{S} and R_{P} in FIG. 12B into R_{S-equ}, and C_{S-equ} in FIG. 12A can be performed using equations (4) and (5). Because R_{S}«R_{P}, the DC bias voltage of 1.75 V appeared almost unchanged, i.e., 1.73 V, on the membrane after collapse. In R-collapse mode, described in this invention, additional benefit is gained as follows:
For a transducer operating in R-collapsed mode, an acoustic pressure might cause more dimples to come into touch (FIG. 11B), a dimple contact to change the electrical conduction on the rim of the changing dimple contact zone, hereby decreasing R_{P}. Taking derivative of equation (4) with respect to R_{P}, we find that

$$\frac{Δ {C}_{S - equ}}{Δ {R}_{P}} = - \frac{2}{{w}^{2} {C}_{S} {R}_{P}^{3}} \qquad ( 6 )$$

capacitance increase will be boosted with the decreasing R_{P} (negative ΔR_{P}.

R-collapse mode enables important features. Dimple resistance (FIG. 12B) is adjusted by the DC bias voltage after collapse. For example, if the DC bias voltage was increased to 2 V, R_{P} was extracted as 11.7 kΩ, which is approximately 20% lower than R_{P} of 15.2 kΩ at DC bias voltage of 1.75 V. If the DC bias voltage was decreased to 1.5 V, R_{P} was extracted as 19.9 kΩ, which is approximately 30% higher than R_{P} of 15.2 kΩ at DC bias voltage of 1.75 V. Furthermore, there is a hysteresis behavior for electrical contact resistance for increasing and decreasing force performed via DC bias sweep. When the DC bias voltage was swept from 0 V up to 2 V in the increasing voltage direction at a constant frequency of 50 kHz, R_{P} was extracted as 15.2 kΩ and 11.7 kΩ at 1.75 V and 2 V, respectively. However, when the DC bias voltage was swept from 2 V down to 0 V in the decreasing voltage direction, R_{P} is extracted as 19.0 kΩ (instead of 15.2 kΩ) and 13.0 kΩ (instead of 11.7 kΩ) at 1.75 V and 2 V, respectively. Existence of hysteresis in R_{P} is in agreement with the hysteresis of electrical contact resistance previously presented in a force sensing study of ECR [33].

Frequency dependency of the input impedance provides additional advantage for detecting signals at a certain frequency, which is suitable to capture pulse modulation frequencies between 50 kHz and 300 kHz in the detection of brain haemorrhage. As previously mentioned and shown in FIGS. 6A-6C, blood size and modulation frequency are related due to the type of interference (constructive or destructive) of the blood-originated ultrasound wave.

### REFERENCES

[1] Feigin, Valery, L., Krishnamurthi R. V, Parmar, P., Norrving, B., Mensah, G. A., Bennett, D. A., Barker-Collo, S., Moran, A. E., Sacco, R. L., Truelsen, T., Davis, S., Pandian, J. D., Mohammad, M. N, Forouzanfar, H. Nguyen, G., Johnson, C. O, Vos, T., Meretoja, A., Murray, C. J. I., Roth, G. A. 2015. “Update on the Global Burden of Ischemic and Hemorrhagic Stroke in 1990-2013: The GBD 2013 Study.”, Neuroepidemiology, 45.3, 161-176.
[2] Chin, J. H., N. Vora. 2014. “The Global Burden of Neurologic Diseases”, Neurology, 83.4, 349-51.
[3] Marler, J. R., Tilley, B. C, Lu, M., Brott, T. G., Lyden, P. C., Grotta, J. C., Broderick, J. P., Levine, S. R., Frankel, M. P., Horowitz, S. H., Haley, E. C., Lewandowski, C. A. ve Kwiatkowski, T. P. 2000. “Early Stroke Treatment Associated with Better Outcome: The NINDS Rt-PA Stroke Study.”, Neurology, 55.11, 1649-1655.
[4] Schellinger, P D., Jansen, O., Fiebach, J. B., Hacke, W. ve Sartor, K. 1999. “A Standardized MRI Stroke Protocol: Comparison with CT in Hyperacute Intracerebral Hemorrhage.”, Stroke, 30.4, 765-768.
[5] Lazebnik, M., Popovic, D., Mccartney, L., Watkins, C. B., Lindstrom, M. J., Harter, J., Sewall, S., Ogilvie, T., Magliocco, A., Breslin, T. M., Temple, W., Mew, D., Booske, J. B., Okoniewski, M., Hagness, S. C. 2007. “A Large-scale Study of the Ultrawideband Microwave Dielectric Properties of Normal, Benign and Malignant Breast Tissues Obtained from Cancer Surgeries.” Physics in Medicine and Biology, 52.20, 6093-6115.
[6] Klemm, M., Craddock, I., Leendertz, J., Preece, A., Benjamin, R. 2008. “Experimental and Clinical Results of Breast Cancer Detection Using UWB Microwave Radar.”, 2008 IEEE

Antennas and Propagation Society International Symposium.
[7] Simon, C. J., Damian E. D., Mayo-Smith, W. W 2005. “Microwave Ablation: Principles and Applications.”, RadioGraphics.
[8] Mitragotri, S. 2005. “Healing Sound: The Use of Ultrasound in Drug Delivery and Other Therapeutic Applications.”, Nature Reviews Drug Discovery, 255-260.
[9] Wells, P. N T. 2006. “Ultrasound Imaging.”, Physics in Medicine and Biology, 51.13.
[10] Ku, Gengve Wang, V. 2001. “Scanning Microwave-induced Thermoacoustic Tomography: Signal, Resolution, and Contrast.”, Medical Physics, 28.1.
[11] Xu, Minghua, Wang, L. V. “RF-induced Thermoacoustic Tomography.”, Proceedings of the Second Joint 24th Annual Conference and the Annual Fall Meeting of the Biomedical Engineering Society, Engineering in Medicine and Biology.
[12] Liu, Shuangli, Zhao, Z., Zhu, X., Wang, Z., Song, J., Wang, B., Gong, Y, Nie, Z., Liu, Q. H. 2016. “Analysis Of Short Pulse Impacting On Microwave Induced Thermo-Acoustic Tomography.”, PIER C Progress In Electromagnetics Research, C 61, 37-46.
[13] Xu, Minghua, Wang, L. V. 2002. “Time-domain Reconstruction for Thermoacoustic Tomography in a Spherical Geometry.”, IEEE Transactions on Medical Imaging, 21.7, 814-822.
[14] Nan, H., Boyle, K. C., Apte, N., Aliroteh, M. S., Bhuyan, A., Nikoozadeh, A., Khuri-Yakub, B. T., Arbabian, A. 2015. “Non-contact thermoacoustic detection of embedded targets using airborne-capacitive micromachined ultrasonic transducers”, Applied Physics Letters, 106.
[15] Duck, F. A. 1990. “Physical Properties of Tissues”. Londra: Academic Press.
[16] The Foundation for Research on Information Technologies in Society. “Database Summary”. http://www.itis.ethz.ch/virtual-population/tissue-properties/database/database-summary/.
[17] ICNIRP. 1998. “ICNIRP Statement on the Guidelines For Limiting Exposure To Time-Varying Electric, Magnetic, And Electromagnetic Fields (Up To 300 Ghz)”, ed: Health Physics Society.
[18] Institute of Electrical and Electronics Engineers. 2005. “Standard for Safety Levels with Respect to Human Exposure to Radio Frequency Electromagnetic Fields, 3 kHz to 300 GHz”, IEEE Standard C95.1.
[19] Institute of Electrical and Electronics Engineers, IEEE. 2004. “Standard for Safety Levels with Respect to Human Exposure to Radio Frequency Electromagnetic Fields, 3 kHz to 300 GHz—Amendment 2: Specific Absorption Rate (SAR) Limits for the Pinna”, C95.1b.
[20] Federal Communications Committee, FCC, Office of Engineering and Technology, OET, Evaluating Compliance. 2001. “FCC Guidelines for Human Exposure to Radiofrequency Electromagnetic Fields”, Bulletin 65, Edition 97-01.
[21] Apte, N., Nikoozadeh, A., Khuri-Yakub, B., Park, K. 2014. “Bandwidth and sensitivity optimization in CMUTs for airborne applications”, IEEE International Ultrasonics Symposium, IUS [serial online], 166-169.
[22] Apte, N., Park, K., Khuri-Yakub, B. 2013. “Experimental evaluation of CMUTs with vented cavities under varying pressure”, 2013 IEEE International Ultrasonics Symposium (IUS) [serial online], 1724.
[23] Apte, N., Park, K., Khuri-Yakub, B. 2012. “Finite element analysis of CMUTs with pressurized cavities”, 2012 IEEE International Ultrasonics Symposium [serial online], 979.
[24] Bayram, B., Haeggstrom, E., Yaralioglu, G. G., Khuri-Yakub, B. T. 2003. “A new regime for operating capacitive micromachined ultrasonic transducers”, IEEE Trans. Ultrason., Ferroelect., Freq. Cont., vol. 50, 1184-1190.
[25] Y. Huang, E. O. Haeggstrom, X. Zhuang, A. S. Ergun, and B. T. Khuri-Yakub, “Capacitive micromachined ultrasonic transducers (cMUTs) with isolation posts,” in Proc. IEEE Ultrason. Symp., Montreal, Canada, vol. 3, pp. 2223-2226, vol. 3, 23-27 Aug. 2004.
[26] P. Dirksen, “Pre-collapsed CMUT with mechanical collapse retention,” WO 2010/097729 A1, 2 Sep. 2010.
[27] Gurun, G., Hochman, M., Hasler, P., Degertekin F. L. 2012. “Thermal-Mechanical-Noise-Based CMUT Characterization and Sensing”, IEEE Trans. Ultrason., Ferroelect., Freq. Cont., vol. 59, no. 6, 1267-1275.
[28] Qibing, Z., Jiyu, G., Min, H., Renfu, L., Mendoza, F. 2016. “Predicting bruise susceptibility of “Golden Delicious” apples using hyperspectral scattering technique”, Postharvest Biology And Technology [serial online], 86-94.
[29] Conger, J. ve Henderson, J. 2012. “Methods for gas detection using stationary hyperspectral imaging sensors” [serial online].
[30] Soydan, H., Koz, A., Duzgun, H., Alatan, A. 2015. “Oil spill determination with hyperspectral imagery: A comparative study”, 3rd Signal Processing And Communications Applications Conference, SIU 2015—Proceedings [serial online], 2404-2407.
[31] Buzzi, J., Riaza, A., Garcia-Melendez, E., Weide, S., Bachmann, M. 2014. “Mapping Changes in a Recovering Mine Site with Hyperspectral Airborne HyMap Imagery (Sotiel, SW Spain)”, Minerals (2075-163X) [serial online], 313-329.
[32] Ma, T., Yu, M., Chen, Z., Fei, C., Shung, K., Zhou, Q. 2015. “Multi-frequency intravascular ultrasound (IVUS) imaging”, IEEE Transactions OnUltrasonics, Ferroelectrics, And Frequency Control [serial online], 97-107.
[33] Rauscher S. G., Bruck H. A DeVoe D. L “Electrical contact resistance force sensing in SOI-DRIE MEMS”, Sensors and Actuators A: Physical, 269 (2018) 474-482.
[34] Kogut L., Komvopoulos K. 2004, “Electrical contact resistance theory for conductive rough surfaces separated by a thin insulating film”, Journal of Applied Physics, vol. 95 (2), 576-585.
[35] Center for Devices and Radiologic Health. 2003. Criteria for significant risk investigations of magnetic resonance diagnostic devices. Rockville, Md.: Food and Drug Administration.
[36] Wang, L. V. 2009. “Multiscale photoacoustic microscopy and computed tomography”, Nature Photonics, vol. 3, 503-509.
[37] Nan, H., Arbabian, A. 2014. “Stepped-frequency continuous-wave microwave-induced thermoacoustic imaging”, American Institute of Physics, Applied Physics Letters.
[38] Bozkurt, A., Yarahoglu, G. G. 2016. “Receive-Noise Analysis of Capacitive Micromachined Ultrasonic Transducers”, IEEE Transactions on Ultrasonics, Ferroelectrics, and Frequency Control, vol. 63.
[39] Wygant, I. O., Kupnik, M., Khuri-Yakub, B. T. 2016. “CMUT Design Equations for Optimizing Noise Figure and Sourse Pressure”, 2016 IEEE International Ultrasonics Symposium Proceedings.
[40] Saffold, J. A., Williamson, F., Ahuja, K., Stein, L., Muller, M. 2016. “Radar-acoustic interaction for IFF applications”, Georgia Tech Research Institute.
[41] Kraftmakher, Y. 2006. “Noise Reduction by Signal Accumulation”, The Physics Teacher, vol. 44.
[42] M. Myers, M. Leidner, H. Schmidt, S. Sachs, A. Baeumer, “Contact Resistance Reduction by Matching Current and Mechanical Load Carrying Asperity Junctions,” 2012 IEEE 58th Holm Conference on Electrical Contacts (Holm), Portland, Oreg., 2012, pp. 1-8.
[43] Tawfik, H. H., Alsaiary, T., Elsayed, M. Y, Nabki, F., El-Gamal, M. N., “Reduced-gap CMUT implementation in PolyMUMPs for air-coupled and underwater applications”, A: Physical, 2019.

What is claimed is:

1. A micro-electro-mechanical system (MEMS) airborne ultrasonic transducer system operating on a thermoacoustic principle to determine brain haemorrhage, comprising:
a radio frequency (RF) transmitter and ultrasound receiver systems to transmit RF energy and receive ultrasound wave, respectively,
an RF transmitter system having an RF signal generator, an RF amplifier and a horn antenna, and
each of the ultrasound receiver systems having a lock-in amplifier, a direct current (DC) supply and two ultrasonic transducer arrays wirebonded to low noise amplifier (LNA) chips.

2. The MEMS airborne ultrasonic transducer system according to claim 1, wherein an RF-induced volumetric expansion of blood in a brain launches the ultrasound wave to be detected with the ultrasound receiver system.

3. The MEMS airborne ultrasonic transducer system according to claim 1, wherein a pulse modulation frequency of the RF transmitter is between 50 kHz and 300 kHz.

4. The MEMS airborne ultrasonic transducer system according to claim 1, wherein a carrier frequency of the RF transmitter is between 1.8 GHz and 2.4 GHz.

5. The MEMS airborne ultrasonic transducer system according to claim 1, wherein human safety levels (<8 W/kg) are not exceeded by a power input of the RF transmitter.

6. The MEMS airborne ultrasonic transducer system according to claim 1, wherein
the ultrasound receiver system comprises two ultrasonic transducer arrays,
each ultrasonic transducer array of the two ultrasonic transducer arrays is wirebonded to one of the LNA chips,
each ultrasonic transducer array is composed of independent four transducers in 2×2 CMUT configuration,
four transducers in each ultrasonic transducer array differ in membrane size to have an incremental difference in a resonance frequency from one another, and
each ultrasonic transducer array supports hyperspectral imaging and enhanced bandwidth modes by changing a DC voltage during operational use.

7. The MEMS airborne ultrasonic transducer system according to claim 6, wherein
each of the four transducers is a capacitive micromachined ultrasonic transducer (CMUT),
each of the four transducers operates in air without touching a subject of interest (i.e., head suspected of having brain haemorrhage),
each of the four transducers has a poly silicon membrane acting as a top electrode,
each of the four transducers has a poly silicon bottom electrode,
each of the four transducers has poly silicon dimples facing the poly silicon bottom electrode,
each of the four transducers has no insulation layer keeping the top electrode and the poly silicon bottom electrode from passing current in-between at membrane collapse,
each of the four transducers has the top and bottom poly silicon electrodes covered by a very thin native oxide (10 Å) enabling a tunneling resistance,
each of the four transducers has an electrical contact resistance (ECR) observed at Hertzian contact of the poly silicon dimples,
lack of insulation layer solves a common charging problem associated with insulators in a high electric field,
each of the four transducers operates reliably at a resistive-collapse (R-collapse) mode,
each of the four transducers utilizes insulator-free, high-resistance (>10 kΩ) Hertzian contact version of collapse mode operation of the CMUT,
a control range of a transducer membrane against ultrasound stimulation and a sensitivity of a measuring system are adjusted by controlling a DC bias voltage after the membrane collapse, and
the DC bias voltage of the transducer membrane is configured to be changed down to a snapback voltage or changed up beyond a collapse voltage.

8. The MEMS airborne ultrasonic transducer system according to claim 7, wherein
a diameter of each of the poly silicon dimples is 8 μm,
a thickness of each of the poly silicon dimples is 0.75 μm,
the poly silicon dimples each have a curved surface profile forming a small-sized Hertzian contact at the membrane collapse,
the poly silicon dimples are spatially distributed on a contacting surface of the transducer membrane,
the poly silicon dimples form the small-sized Hertzian contact with the poly silicon bottom electrode at the membrane collapse, and
the poly silicon dimples present a high electrical resistance at the membrane collapse.

9. The MEMS airborne ultrasonic transducer system according to claim 7, wherein specifications of each of the four transducers are:
collapse voltage is 1.4 V,
snapback voltage is 1.25 V,
impedance model parameters R_{S}, C_{S} and R_{P} are 150 Ω, 36.7 pF and 15.2 kΩ at the DC bias voltage of 1.75 V, respectively,
the DC bias voltage applied on the poly silicon membrane is almost unchanged at the R-collapse mode since R_{S} is much smaller than R_{P},
each of the four transducers features broad bandwidth and high sensitivity (i.e., high displacement response) at the R-collapse mode, i.e., collapse mode with the ECR.

10. The MEMS airborne ultrasonic transducer system according to claim 1, wherein operates as follows:
the RF signal generator generates a pulse modulated RF carrier signal,
the RF signal generator sweeps a pulse modulation frequency from 50 kHz up to 300 kHz,
the RF signal generator is connected to the lock-in amplifier for sync,
an DC bias voltage of each of the two ultrasonic transducer arrays is adjusted for maximum sensitivity for a present pulse modulation frequency,
the lock-in amplifier tracks the pulse modulation frequency,
the lock-in amplifier measures a signal coming from the LNA chips to calculate a spectral ultrasound power at a predetermined frequency for a specific blood size to benefit from constructive and destructive interference of RF-induced blood-originating ultrasound waves,
the lock-in amplifier uses a time-gated mode to process only a predetermined time waveform interval between t_{START} and t_{STOP} (referenced to a trigger signal from the RF signal generator) determined from an ultrasound time-of-flight calculation for a certain region within a brain,
lock-in amplifier data collected from #1 MEMS ultrasonic transducer and #2 MEMS ultrasonic transducer, each having 4 units (CMUT #1 to CMUT #4), are processed with multi-frequency and multi-band (hyperspectral) imaging techniques,
equipments for the RF transmitter and the ultrasound receiver systems are controlled by a personal computer and a software, and
frequency domain analysis of thermoacoustic ultrasound wave caused by blood accumulation of certain size under RF energy transfer is performed.
